(19) United States
(12) Patent Application Publication
(10) Pub. No.: US 2002/0169438 A1
(43) Pub. Date: Nov. 14, 2002

(54) Guide device for introducing a catheter into the urethra

(76) Inventor: Manfred Sauer, Lobbach (DE)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 10/189,609

(22) Filed: Jul. 3, 2002

Related U.S. Application Data
(63) Continuation of application No. PCT/DE01/00004, filed on Jan. 4, 2001.

(30) Foreign Application Priority Data
Jan. 5, 2000 (DE) 100 00 267.6
Mar. 24, 2000 (DE) 100 14 857.3

(51) Int. Cl. A61M 25/00

(52) U.S. Cl. 604/544

(57) ABSTRACT

A device for inserting a tubular catheter (1) into the urethra, preferably for catheterizing male persons, in particular in the case of paraplegia. The device comprises a guide means (2) for inserting the catheter (1), with the guide means composed of an outer funnel (3) that is intended for insertion into the urethra orifice, and an inner funnel (4) that is adapted for insertion into the urethra through the outer funnel (3). The catheter (1) is then insertable into the urethra through the inner funnel (4).

### CROSS REFERENCE TO RELATED APPLICATION

This is a continuation of international application PCT/DE01/00004 filed Jan. 4, 2001, and designating the U.S.

### BACKGROUND OF THE INVENTION

The invention relates to a guide device for introducing a catheter into the urethra, preferably for catheterizing male persons, in particular in the case of paraplegia.

For managing incontinence of male persons, in particular in the case of paraplegia, one applies, among other things, the so-called intermittent selfcatheterization (ISK), by which the incontinent male person catheterizes himself four to six times a day. To remain continent between the catheterization phases, the treating physician normally prescribes a medication that reactivates or deactivates the bladder.

However, presently known catheters for withdrawing urine from the bladder are problematic in practice, inasmuch as during the introduction of the catheters, bacteria that have externally settled on the tip of the glans penis or in the front region of the urethra are conveyed inward into the urethra. In any case, even when applying the best hygiene, the catheters of the prior art barely permit guiding the catheter through the germ affected zone free of germs as far as into the bladder.

To prevent the urethra or bladder from being additionally exposed to germs by the catheterization procedure, it has until now been common practice to use high doses of a disinfecting lubricant for the catheter. In this connection, the side effects of the lubricant and disinfecting additives have not yet been researched to a great extent.

Also, for the purpose of avoiding entraining germs with the catheter during the insertion process, it has been known to employ an approximately 15 mm long connecting piece which is initially inserted into the urethra orifice, and through which the actual catheter is introduced into the urethra. However, this procedure likewise is unable to avoid in a reliable manner that germs are also conveyed via this connecting piece farther into the urethra, and then enter the bladder through the urethra with the aid of the catheter.

Before the actual catheterization, it is commonly recommended to disinfect the glans penis head, for example, with swabs. While a patient has adequate time in the course of his hospital stay for such a disinfecting measure, the time required therefor after the hospital stay is not always acceptable. Consequently, the recommended amount of hygiene and therefor necessary disinfection of the affected person are reduced, and the tip of the glans penis undergoes disinfection by spraying. Likewise in this instance, the necessary time to take effect falls short, in most cases for lack of time.

Lastly, in particular in the case of selfcatheterization, one always has to start out from the fact that the tip of the glans penis and the urethra orifice are most affected by germs, and that systems of the prior art always carry the risk that germs having settled there are conveyed through the urethra into the bladder. Inflammations or infections are the consequence.

It is therefore an object of the present invention to provide a device for catheterizing male persons, which permits performing catheterization under the best possible hygienic conditions in a time efficient manner.

### SUMMARY OF THE INVENTION

The foregoing object is accomplished by a guide means or which comprises an outer funnel, which is intended for insertion into the orifice of the urethra, and an inner funnel adapted for insertion into the urethra through the outer funnel, with the catheter then being introduced into the urethra through the inner funnel.

In accordance with the invention, it has been recognized that the connecting piece that has been used until now in the art, does not suffice to prevent germs from being conveyed through the urethra into the bladder. Furthermore, it has been recognized that one can counteract the problem arising in the state of the art in that the guide means of the invention is made quasi telescopic, namely by combining an outer funnel that is intended for being inserted into the urethra orifice, with an inner funnel that can be inserted through the outer funnel farther into the urethra. Both funnels form a device operating in the fashion of a telescope, with the outer funnel initially operating like a connecting piece known from the art, and the inner funnel being inserted into this outer funnel, and entering the urethra with its front end extending beyond the outer funnel. Only thereafter, without touching the tip of the glans penis, the urethra, or the outer funnel, is the catheter introduced through the inner funnel into the urethra, so that catheter does not come into contact with the germ-affected areas at all. As a result, it is accomplished in accordance with the invention that the guide means forms a telescope-type sluice, so that conveyance of germs to the bladder is avoided to the greatest extent. Furthermore, the device of the present invention with the guide means provided therein ensures a simple and yet reliable handling, inasmuch as the guide means is used not only for preventing conveyance of germs, but also predetermines a kind of restricted guidance for the actual catheter. In this respect, the risk of injury during the selfcatheterization is quite considerably reduced.

In the preferred embodiment, the outer funnel includes an annular rim, which limits the insertion into the urethra, and covers in the inserted state the urethra orifice or the tip of the glans penis. This rim covers the urethra orifice in the fashion of an umbrella, so that after the insertion of the outer funnel, the area that is occupied by bacteria or germs is covered, while avoiding a more extensive contacting. To this end, the rim may be arcuately curved so as to be adapted in its shape at least largely to the tip of the glans penis or the glans penis.

In a further advantageous manner, the inner funnel is similarly constructed. It includes likewise an annular rim, which limits the insertion into the urethra and reaches in its inserted state the rim of the outer funnel and comes into contact with the inner side thereof. In this connection, the inner funnel is made axially longer than the outer funnel, so that the inner funnel can be inserted with its inner end through the outer funnel into the urethra. For example, the outer funnel could have a length of 15 mm and the inner funnel a length of 40 mm, so that the inner funnel can be inserted into the urethra without directly contacting the tip of the glans penis or the orifice of the urethra. Only after the inner funnel is completely inserted, is the catheter introduced through the inner funnel into the urethra.

As regards a concrete realization of the inner funnel, it should be added that the rim of the inner funnel may be approximately adapted to the shape of the inner side of the outer funnel, so that the inner funnel can be inserted as far as possible, until the two rims mutually abut each other, and is yet prevented from sliding therethrough.

In their sterilized or germfree state, the two funnels may also be provided with a lubricant. To this end, the outer funnel could be closed at its inner end that is used for insertion into the urethra orifice, and made frangible so that it can be opened, when the inner funnel is pushed therethrough. Likewise, it would be possible that the inner funnel is initially closed at its inner end that is used for the insertion into the outer funnel, and it is also frangible so that it can be opened when the catheter is inserted.

The closure that seals the two funnels, could be constructed such that the inner ends of the funnels are slotted in a way similar to predetermined breaking points, preferably in the form of a cross. Specifically, the inner ends of the funnels could be made in a budlike configuration. This will cause the funnels to open outward during the occurrence of an inner resistance.

As has previously been described, the initial closure of the two funnels serves not only to preserve sterile conditions, but it also enables the formation of a reservoir within each funnel, which stores a lubricant. Thus, both the front portion of the outer funnel and the front portion of the inner funnel could serve as a reservoir for a lubricant. This results in an especially advantageous effect in that during the insertion of the inner funnel, the outside wall of the inner funnel is wetted with the lubricant, and only then comes into contact with urethra. The situation is similar during the subsequent insertion of the catheter, which is pushed through the lubricant reservoir of the inner funnel, and becomes wetted with the lubricant, and then enters the urethra.

Furthermore, it is desirable to have the outer funnel externally wetted with a lubricant, so that it also can be easily inserted into the orifice of the urethra.

The lubricant may be commercially available lubricants, which are recommended for catheterization, for example oil, Vaseline™, or the like. In a very advantageous manner, a disinfectant may be added to the lubricant, so that likewise this measure avoids as much as possible that germs are carried in. It would likewise be possible to add to the lubricant a medication or narcotic depending on need and indication.

In an advantageous manner, the funnels are made of a preferably soft plastic. In this connection, the use of silicone is particularly well suited. The funnels may be constructed as injection molded parts, with the inner funnel being made at least slightly firmer and harder than the outer funnel.

The operation of the guide means has already been described. For a simple handling, the device could be offered in an assembled state of the guide means. In this instance, the inner funnel is partially inserted into the outer funnel. Both funnels are closed at their inner ends, so that lubricants may be stored in the resulting reservoirs. Furthermore, the outer funnel may be likewise wetted with the lubricant on its outside wall, so that the entire guide means can be handled without requiring an additional lubricant, and without requiring more extensive auxiliary means.

For obtaining sterile conditions, the inner funnel may be initially sealed at its rear end, i.e. the inlet side, with the outer funnel in turn being closed at least as much as possible by the inner funnel. The closure of the inner funnel could be realized as a strippable film, a plug, or the like. Moreover, it would be possible to provide the closure with a strap for handling and opening, or with a corresponding string, a tape, or the like, so that handling is also simplified in this respect.

Moreover the assembled guide means can be packaged free from germs in a sterile container. The container could be adapted at least largely to the shape of the entire guide means, i.e., both funnels. Likewise, the container in turn could include a reservoir for a lubricant. In this case, the outer funnel is stored in the reservoir with its inner end or portion that is used for insertion into the urethra, so that the outside wall of the outer funnel is always wetted with a lubricant, disinfectant, and/or a medicated additive.

The container for accommodating the guide means could be made of plastic, so that in this instance a deep drawn plastic part can be used as the container. The container could be sealed by a strippable film, or with a plug or lid, so that the entire guide means is always made available to the user, free from germs, in a sealed container. Handling turns out to be extremely simple, in that one opens the container, removes the already partially assembled funnels, and inserts the outer funnel into the urethra orifice until its rim abuts. Since the outer area is externally coated at least in the lower portion with a disinfecting lubricant, the introduction of germs is already limited in this respect.

After inserting the outer funnel into the urethra orifice, the inner funnel is caused to follow through the lubricant reservoir into the urethra. Likewise with that, a farther reaching introduction or conveyance of germs is prevented, inasmuch as this lubricant may also be provided with disinfectants. Finally, the orifice of the inner funnel is opened by stripping the film or the like, so that, when the rim of the inner funnel abuts the rim of the outer funnel, it is possible to insert the catheter. During this insertion process, the catheter becomes automatically wetted within the lubricant stored in the lower portion of the inner funnel, so that a reliable introduction and a prevention of a conveyance of germs are likewise realized to this extent. The further catheterization occurs as usual.

There exist various possibilities of improving and further developing the teaching of the present invention in an advantageous manner. To this end, one may refer to the following description of a preferred embodiment of the invention, with reference to the drawings. Also generally preferred improvements and further developments of the teaching are explained.

### BRIEF DESCRIPTION OF THE DRAWINGS

In the drawings:

FIG. 1 is a schematic side view of an embodiment of a device according to the invention, with the parts being separated;

FIG. 2 is a schematic side view of the subject matter of FIG. 1 in a preassembled condition, i.e. with the funnels engaging each other;

FIG. 3 is a schematic side view of the subject matter of FIGS. 1 and 2 with a fully inserted inner funnel and with an indicated catheter;

FIG. 4 is a schematic side view and bottom view of the free ends of each of the funnels with desired breaking points; and

FIG. 5 is a schematic side view of a container for accommodating the device of FIGS. 1-4.

### DETAILED DESCRIPTION OF THE PREFERRED EMBODIMENT

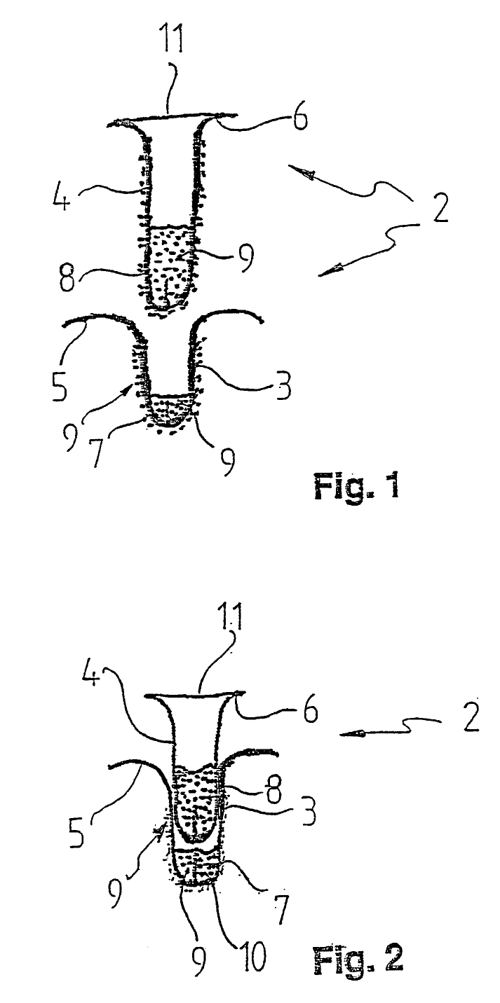

FIGS. 1-4 illustrate an embodiment of a guide device according to the invention, for introducing a tubular catheter 1 into the urethra, namely for catheterizing male persons. In particular, the device is used for cases of paraplegia. The device comprises a guide means 2 for inserting the catheter 1 into the urethra.

In accordance with the invention, the guide means 2 includes an outer tubular funnel 3, which is intended for insertion into the urethra orifice, and an inner tubular funnel 4, which is adapted for insertion into the urethra through outer funnel 3. The catheter 1 is inserted through inner funnel 4 into the urethra.

As best seen in FIGS. 1-3, the outer funnel 3 includes an annular rim 5, which limits insertion into the urethra, and covers in its inserted state the urethra orifice or the tip of the glans penis. With its arcuate shape, the rim 5 is adapted to the tip of the glans penis or the glans penis.

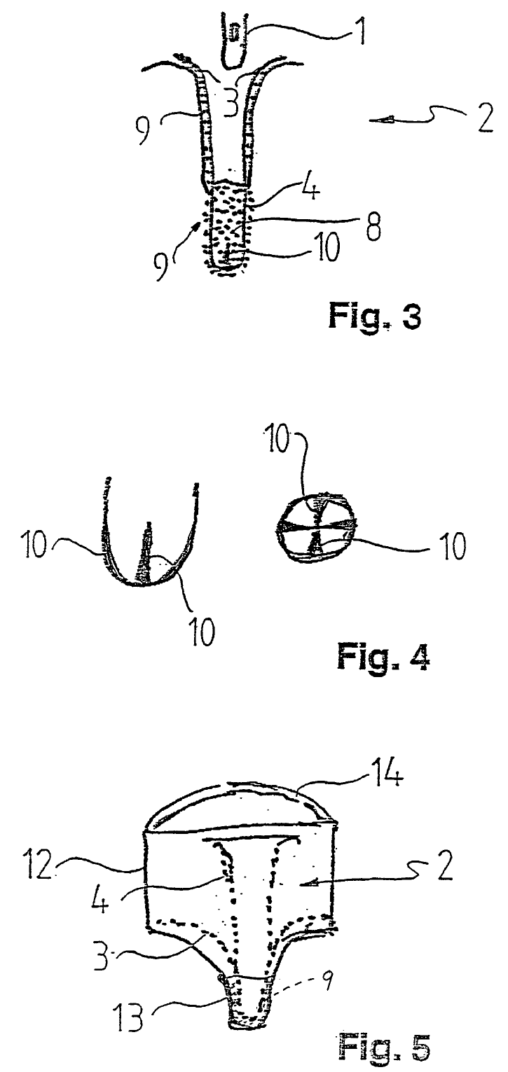

The same applies to the inner funnel 4, which includes an annular rim 6 that limits insertion into the urethra and comes to abut rim 5 of outer funnel 3 on the inner side thereof. As best seen in FIG. 3, the rim 6 of inner funnel 4 is approximately adapted to the shape of the inner side of rim 5 of the outer funnel 3.

As best seen in FIGS. 1-3 in combination with FIG. 4, both the outer funnel 3 and the inner funnel 4 are each closed at their inner ends and can be opened, respectively when sliding therethrough the inner funnel 4 and inserting the catheter 1. To this extent, the inner or lower ends of funnels 3, 4 form reservoirs 7, 8 for accommodating a lubricant 9. This lubricant 9 may optionally be mixed with a disinfectant or narcotic.

As best seen in FIG. 4, the inner ends of funnels 3, 4 are scored in the shape of a cross so as to be frangible and resulting in a budlike configuration when broken. The there provided desired breaking points 10 enable an opening of the respective funnel 3, 4, when inserting inner funnel 4 or sliding therethrough catheter 1. In so doing, the lubricant 9, if need be, including the disinfectant are released, so that funnel 4 having been passed therethrough and catheter 1 being inserted therein, are each adequately wetted on their outer side with the lubricant 9.

As further indicated in FIGS. 1-3, the outer funnel 3 is likewise wetted on its outside wall with the lubricant 9 for purposes of enabling insertion or introduction of outer funnel 3, while preventing bacteria from being entrained.

In a selected embodiment, the two funnels 3, 4 are made of silicone, and they may be injection-molded parts. The inner funnel 4 is made at least slightly firmer or harder than the outer funnel 3, thereby simplifying likewise the handling, while reducing a risk of injury. At this point, it should be remarked that for the production of the two funnels 3, 4 any suitable materials and corresponding production methods may be considered.

FIG. 2 shows the guide means 2 in its partially assembled state, wherein the inner funnel 4 is somewhat inserted into the outer funnel 3. The two reservoirs 7, 8 are adequately filled with lubricant 9, with the outside wall of outer funnel 3 being likewise provided with lubricant 9.

As further shown in FIG. 2, the inner funnel 4 is sealed at its rear end by means of a strippable film 11. As an alternative thereto, it would also be possible to provide a lid or any other closure of any form. In the selected embodiment, the film 11 is secured by an adhesive to the outer rim 6 of inner funnel 4, and can be removed by a simple stripping.

In the illustration of FIG. 3, the inner funnel 4 has been completely inserted into outer funnel 3. The film 11 is removed, and the catheter is indicated shortly before its insertion.

As roughly shown in FIG. 5, the assembled guide means 2 is made available free from germs in a sterile container 12. This container 12 is adapted in its shape to the guide means 2. With its inner end or with its lower portion that is used for the insertion, the outer funnel 3 is stored in a there provided reservoir 13. This reservoir 13 also contains the lubricant 9. With that, it is ensured that the outside wall of outer funnel 3 is adequately supplied with lubricant 9.

The container 12 may be made of plastic. In the selected embodiment, the container is a deep drawn plastic part. For a germfree storage, the container 12 is sealed with a strippable film 14. It is also possible to provide in the place of film 14, a lid, a plug, or the like.

The device of the invention has the advantage of simplified storage and use in the hospital, in the retail trade, as well as at home. A kind of modular solution results in particular in that guide means and catheters of different sizes and different diameters are freely combinable. Lastly, above and beyond conventional catheters and other disinfectants, it can be made available, packaged free from germs, in a sterile container.

As regards the operating mode as well as a more extensive description, the description of the background of the invention is herewith incorporated by reference for purposes of avoiding repetition.

Finally, it should be expressly stated that the above-described embodiment is used to explain only the teaching of the invention, without however limiting it to the disclosed embodiment.

1. A guide device for the introduction of a catheter into the urethra, comprising
an outer funnel which is sized for insertion into the urethra orifice, and
an inner funnel which is sized for insertion through the outer funnel and into the urethra, and so that a catheter is then insertable into the urethra through the inner funnel.

2. The guide device of claim 1, wherein the outer funnel includes an annular rim which limits insertion into the urethra, and covers in its inserted state the urethra orifice or tip of the glans penis.

3. The guide device of claim 2, wherein the inner funnel includes an annular rim which limits insertion into the urethra, and which comes into contact in its inserted state with the rim of the outer funnel.

4. The guide device of claim 3, wherein the annular rim of the inner funnel is approximately adapted to the shape of the annular rim of the outer funnel.

5. The guide device of claim 1, wherein the outer funnel is closed at an inner end that is used for insertion into the urethra orifice, and said inner end is configured to be opened when the inner funnel is inserted through the outer funnel.

6. The guide device of claim 5, wherein the inner funnel is closed at an inner end that is used for insertion into and through the outer funnel, and the inner end of the inner funnel is configured to be opened during the insertion of the catheter.

7. The guide device of claim 6 wherein the inner ends of the inner and outer funnels are each slotted to define desired breaking points.

8. The guide device of claim 7, wherein a front portion of the outer funnel serves as a reservoir for a lubricant.

9. The guide device of claim 8, wherein a front portion of the inner funnel serves as a reservoir for a lubricant.

10. The guide device of claim 1, wherein the outer funnel is externally wetted with a lubricant.

11. The guide device of claim 10, wherein the lubricant includes a disinfectant and/or a medication.

12. The guide device of claim 1, wherein the inner and outer funnels are made of a relatively soft plastic material.

13. The guide device of claim 12, wherein the plastic material of the outer funnel is somewhat softer than that of the inner funnel.

14. The guide device of claim 1, wherein the inner funnel has an outer end which is opposite the end which is inserted into and through the outer funnel, and said outer end is closed with a removable closure.

15. The guide device of claim 14, wherein the closure is provided with a loop that is used for handling and opening, or with a corresponding strap or string.

16. A guide device for the introduction of a catheter into the urethra, comprising
an outer tubular funnel having a frangible closed inner end and an open outer end, and which has a size and length to permit the inner end to be inserted into the urethra orifice, and
an inner tubular funnel having a frangible closed inner end and an outer end, and having a length greater than that of the outer funnel and a size to permit the inner funnel to be inserted through the frangible closed inner end of the outer funnel with the inner end of the inner funnel extending beyond the outer funnel and into the urethra, and so that a catheter may then be inserted through the frangible closed inner end of the inner funnel and into the urethra.

17. The guide device of claim 16, wherein the inner funnel is partially assembled in the outer funnel, and wherein a lubricant is retained in the closed inner end of the outer funnel and in the closed inner end of the inner funnel.

18. The guide device of claim 17, wherein the outer funnel has an outer surface which is coated with a lubricant.

19. The guide device of claim 17, wherein the outer end of the inner funnel is closed by a removable closure.

20. An assembly comprising
a container,
a guide device for the introduction of a catheter into the urethra, comprising an outer funnel which is sized for insertion into the urethra orifice, and an inner funnel which is sized for insertion through the outer funnel and into the urethra, and so that a catheter is then insertable into the urethra through the inner funnel, and
said guide device being packaged in said container under sterile conditions.

21. The assembly of claim 20, wherein the inner funnel is partially assembled in the outer funnel, and wherein the container is adapted at least largely to the shape of the partially assembled inner and outer funnels.

22. The assembly of claim 20, wherein the container comprises a reservoir containing a lubricant, and wherein the outer funnel is packaged with an inner end portion that is used for insertion immersed in the lubricant reservoir.

23. The assembly of claim 20 wherein the container is sealed with a strippable film or the like, or with a lid or a plug.
